(19) United States
(12) Patent Application Publication
(10) Pub. No.: US 2004/0126428 A1
(43) Pub. Date: Jul. 1, 2004

(54) Pharmaceutical formulation including a resinate and an aversive agent

(76) Inventors: Lyn Hughes, Harleysville, PA (US); Simon Andrew Bellamy, Redhill (GB)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 10/713,926

(22) Filed: Nov. 14, 2003

Related U.S. Application Data
(63) Continuation-in-part of application No. 10/016,336, filed on Nov. 2, 2001.

(51) Int. Cl. A61K 9/24

(52) U.S. Cl. 424/471

(57) ABSTRACT

The present invention provides a pharmaceutical that includes, in combination, a core, and a coating surrounding the core comprising a resinate of an opiate. The pharmaceutical oral dosage form is failsafe, and not subject to abuse.

This application claims benefit to U.S. patent application Ser. No. 10/016,336, filed Nov. 2, 2001, entitled: “Dosage Forms.”

Abuse of controlled substances is a serious and growing problem throughout the world. There are three main routes that drug abusers use for administering the drug substances: parenteral, oral, and inhalation. The parenteral route is commonly called ‘mainlining’ and requires the drug substance to be in solution such that it can be injected intravenously with a syringe. For solid dosage form drugs this requires some type of extraction and concentration procedure to render the drug substance suitable for injection. Inhalation of a solid drug substance through the nose is commonly called ‘snorting’. For solid dosage form drugs this requires only that the dosage form be crushed into a powder, or emptied from a capsule. Breathing in vapors is frequently known as ‘huffing’. Both snorting and huffing result in the rapid absorption of the drug substance through the mucosa of the respiratory system.

The intensity of the drug-induced high depends on the rate of entry of the drug into the blood. Increasing the rate of entry increases the intensity of the drug-induced high. Consequently, injection of the drug directly into the blood gives the most rapid rate of entry, and the most intense drug-induced high. For other routes of administration the route of entry into the blood is by absorption through mucosal membranes.

The potential for abuse is increased by the use of extended release formulations because they typically contain more than the immediate release single dose of active ingredient. Circumventing the extended release mechanism delivers the full dose immediately. For example, crushing an extended release oxycodone tablet that uses a gelling matrix to control the release rate of the oxycodone separates the gelling matrix from the oxycodone active ingredient, such that when the crushed formulation is inhaled through the nose the gelling matrix cannot exert the extended release effect. Similarly it is sometimes possible to circumvent the extended release effect by chewing the dosage form. Such formulations are also susceptible to abuse by illicit extraction.

A further example of an extended release formulation that can be abused is described in U.S. Pat. No. 5,378,474. which is disclosed a formulation comprising a water soluble opiate salt on the surface of a water soluble core that is then coated to retard the dissolution of the water soluble opiate salt. Crushing the coated particles disrupts the release rate controlling coating such that the opiate is released rapidly instead of over an extended period. A similar example using a water soluble salt of an opiate is disclosed in U.S. Pat. No. 6,066,339. In this example, the water soluble salt of the opiate is subject to abuse by the same method of crushing. Similarly it is possible to circumvent the extended release effect by chewing the dosage form. Such formulations are also susceptible to abuse by illicit extraction.

Extended release formulations present a greater hazard to abusers than immediate release formulations because they generally contain larger dosages of the active ingredient. However, immediate release dosage forms are also susceptible to abuse by methods similar to those for the extended release formulations.

A further method of abuse is used for dosage forms that contain two active ingredients, one an opiate and the other a non-opiate. Examples of these dosage forms include Percocet™ (oxycodone hydrochloride combined with acetaminophen), Vicodin™ (hydrocodone bitartrate combined with acetaminophen), and Azdone™ (hydrocodone bitartrate combined with aspirin). There are many different commercially available dosage forms of this type. Over 90 of these dosage forms exist in the USA alone. The dosage forms are abused by extracting the opiate in such a way as to separate it from the bulk of the non-opiate active ingredient. This illicit extract is then administered orally, rectally, or by injection.

In yet another method of abuse, a transdermal patch comprising the opiate fentanyl (sold commercially as Duragesic™) is abused by the illicit extraction of the active ingredient which is then administered orally, rectally, or by injection. In yet a further method of abuse for this dosage form the extended release mechanism can be circumvented by removing the adhesive fabric and sucking on the pellet that remains. Because some of the active ingredient remains in the patch after therapeutic use, both of the methods of abuse can also be used on patches that have been used therapeutically.

In U.S. Provisional Patent Application No. 20030064122 A1, the use of aversive agents is disclosed. These dosages forms provide limited reduction in the potential for abuse. They have no mechanism acting against injection or rectal administration and abuse. They are also not effective against extraction of the opiate from the opiate—nonopiate combination described above. They depend solely on the subjective perception of the abuser as to what is and is not palatable. It is also possible for hard core abusers to develop a tolerance to aversive agents, and thus circumvent the anti-abuse mechanism of this dosage form.

There exists a need in the art for a pharmaceutical dosage form that will greatly reduce the potential for drug abuse by all of the known routes of abuse and is not solely dependent on the subjective perception of the abuser. There also exists a need in the art for a fail-safe oral dosage form that is not susceptible to different techniques for preventing abuse.

Applicants have discovered that the use of a resinate of the active ingredient in an oral dosage form has two effects on the potential for abuse of the dosage form. One effect is to reduce the efficiency of illicit extraction, resulting in less drug being available for administration. This will have the effect of reducing the intensity of the drug-induced high relative to other dosage forms that do not contain a resinate. Another effect of the use of the resinate is to reduce the rate at which the drug enters into solution after administration. The rate at which a drug enters into solution is frequently called the release rate, or dissolution rate of the drug. For a drug to be absorbed into the blood via a mucosal membrane it must first be in solution. The slow release of the drug from the resinate has the effect of reducing the rate of absorption into the blood, which reduces the intensity of the drug-induced high.

The term “therapeutic concentration” as used herein means the concentration of the pharmaceutically active ingredient in the blood plasma that is obtained by the administration of the recommended doses using the prescribed method of administration. Recommended doses for Schedule II-V controlled substances are defined in the literature. For example, see ‘Drug Facts and Comparisons’, published by Facts and Comparisons, St Louis.

The term “drug-induced high,” as used herein means the non-therapeutic effect desired by drug addicts and recreational drug users

The term “mucosal membrane” as used herein means any mucosal membrane of the body through which an active substance can be administered, including, but not limited to, nasal, lingual, buccal, pharyngeal, bronchial, rectal, urethral and vaginal.

The term “respiratory mucosal membrane” as used herein means the mucous membrane lining the nasal and pharyngeal cavities, the bronchial tubes, and the lungs.

The term “sensory agent” as used herein means those agents that modify ones sensory perception of the dosage form.

The term “respiratory irritant’ as use herein means substances that cause irritation when administered to the respiratory mucosal membrane. The irritation can include, but is not limited to, coughing, dyspnea, rhinitis, nasal congestion, eye irritation, lachrymation, and sneezing.

When describing dosage forms the term “immediate release” as used herein means a dosage form from which the active ingredient is dissolved as quickly as possible after administration. In the pharmaceutical arts the immediate release dosage forms are frequently referred to as “conventional” dosage forms.

When describing dosage forms, the term “modified release” as used herein means a dosage form whose drug-release characteristics of time course and/or location are chosen to accomplish therapeutic or convenience objectives not offered by conventional dosage forms. The modified release dosage forms include dosage forms commonly known in the art as, delayed, taste-masked, sustained, extended, targeted, prolonged, pulsatile, zero-order, constant rate, and controlled.

The term “aversive response” as used herein means a response in a person, resulting from an act that is sufficiently unpleasant that the person decides not to perform the act again.

The term “aversive agent” as used herein means any substance that is included in a dosage form that creates an aversive response.

The term “nociceptive” as used herein means a response characterized by pain. For example the term ‘nociceptive efficacy’ when applied to an irritant refers to the quantification of the ability of the irritant to cause pain.

The term “illicit extracts” as used herein are those extracts obtained by any of the means known to drug addicts, drug users, and recreational drug users for extracting an active substance from an oral dosage form that contains a single active ingredient or multiple active ingredients. In the interests of social responsibility these methods will not be described herein.

The term “meq/g”, as used herein, refers to the fact that ion exchange resins are characterized by their capacity to exchange ions. This is expressed as the “Ion Exchange Capacity.” For cation exchange resins the term used is “Cation Exchange Capacity,” and for anion exchange resins the term used is “Anion Exchange Capacity.” The ion exchange capacity is measured as the number equivalents of an ion that can be exchanged and can be expressed with reference to the mass of the polymer (herein abbreviated to “Weight Capacity”) or its volume (often abbreviated to “Volume Capacity”). A frequently used unit for weight capacity is “milliequivalents of exchange capacity per gram of dry polymer.” This is commonly abbreviated to “meq/g.”

In one variant, the present invention provides a pharmaceutical dosage form that includes, in combination, a resinate and an aversive agent. The resinate includes an ion exchange resin and a drug. The drug is a controlled substance. In variants of the invention, both the aversive agent and the controlled substance are loaded onto the ion exchange resin; the aversive agent is loaded onto the ion exchange resin, and the controlled substance is not loaded onto the ion exchange resin; the controlled substance is loaded onto the ion exchange resin, and the aversive agent is not loaded onto the ion exchange resin; or, the controlled substance is loaded onto a first ion exchange resin, and the aversive agent is loaded onto an ion exchange resin different from the first ion exchange resin. The resinates of the current invention are not soluble in water.

By way of example, the present invention provides an oral pharmaceutical dosage form comprising, in combination, a core, and a coating surrounding the core comprising a resinate of an opiate. The pharmaceutical oral dosage form is not subject to abuse, and thus is a fail safe oral dosage form. The coating is an extended drug release coating in one variant, and the coating on the core can comprise the following components: (a) from 1 to 85% by weight of a matrix polymer which is insoluble at a pH of from 1 to 7.5 and contributes to the control of the rate of release of the active ingredient in the stomach and intestines; (b) from 1 to 30% of an enteric polymer which is substantially insoluble at a pH of from 1 to 4, sufficient to delay the release of the active ingredient in the stomach, but which is soluble at a pH of from 6 to 7.5 so as not to substantially delay release in the intestines; (c) from 1 to 60% of a compound soluble at a pH of from 1 to 4, sufficient to enable initiation of release of the active ingredient in the stomach; the percentages being by weight based on the total weight of components (a), (b), and (c); the ratio of the components (a), (b), and (c) in the coating being such that a dose of the pellet composition delivers to the patient a therapeutically effective amount of the active ingredient over the course of the predetermined interval, so as to maintain an active ingredient blood level at steady state of at least 75% of maximum blood level for more than approximately 4 hours and so that the time at which the active ingredient reaches its maximum concentration is between about 4 and about 30 hours.

In a further variant, the formulation of active, alone or in combination with the other dosage form ingredients, is formulated so that the active ingredient reaches its released maximum concentration in a subject in between about 4 hours to about 12 hours. The opiate is selected from the group consisting of codeine, dextromoramide, hydrocodone, hydromorphine, pethidine, methadone, morphine, oxycodone, difydrocodeine, fentanyl, and propoxyphene.

In another variant of the invention, the coating comprises: as component (a), ethyl cellulose, a quaternary ammonium acrylic or methacrylic polymer, an acrylic or a methacrylic ester copolymer or a mixture thereof; as component (b), cellulose acetate phthalate, hydroxypropyl methylcellulose phthalate, polyvinyl acetate phthalate, methacrylic acid:acrylic acid ester copolymer, hydroxypropyl methylcellulose acetate succinate, shellac, cellulose acetate trimellitate and mixtures thereof; and as component (c), polyvinylpyrrolidone, hydroxypropyl cellulose, hydroxypropyl methylcellulose, polyethylene glycol having a molecular weight of from 1700 to 20,000, polyvinyl alcohol and monomers therefor and mixtures thereof.

In yet a further variant, the coating comprises: 35 to 75% by weight of component (a); 2-20% by weight of component (b); and 15-40% by weight of component (c), and optionally, comprises up to 50% of plasticizer selected from diethyl phthalate, triethyl citrate, triethyl acetyl citrate, triethyl acetin, tributyl citrate, polyethylene glycol having a molecular weight of from 200 to less than 1700 or glycerol and up to 75% of a filler selected from silicon dioxide, titanium dioxide, talc, alumina, starch, kaolin, polacrilin potassium, powdered cellulose and microcrystalline cellulose and mixtures thereof. The percentages are based on the total weight of the coating. In yet another variant, the dosage form contains: component (a) 35 to 70%; component (b) 4 to 20%; component (c) 15 to 35%; and, plasticizer 4 to 30%.

Optionally, the core comprises as the active ingredient a therapeutically effective amount of a resinate of morphine and the coating on the core which comprises the following components: (a) from 1% to 85% by weight of a matrix polymer which is insoluble at a pH of from 1 to 7.5 and contributes to the control of the rate of release of the active ingredient in the stomach and intestines; (b) from 1 to 30% of an enteric polymer which is substantially insoluble at a pH of from 1 to 4, sufficient to delay the release of the active ingredient in the stomach, but which is soluble at a pH of from 6 to 7.5 so as not to substantially delay release in the intestines; (c) from 1 to 60% of a compound soluble at a pH of from 1 to 4, sufficient to enable initiation of release of the active ingredient in the stomach; the percentages being by weight based on the total weight of components (a), (b), and (c). The ratio of the components (a), (b), and (c) in the coating being are such that a dose of the pellet composition delivers to the patient a therapeutically effective amount of the active ingredient over the course of the predetermined interval, so as to maintain an active ingredient blood level at steady state of at least 75% of maximum blood level for more than approximately 4 hours and so that the time at which the active ingredient reaches its maximum concentration is between about 4 and about 30 hours. It is appreciated that the composition, in use, minimizes fluctuations in the morphine compound concentration in the plasma of a patient.

In one variant of the invention, the coating comprises: as component (a), ethyl cellulose, a quaternary ammonium acrylic or methacrylic polymer, an acrylic or a methacrylic ester copolymer or a mixture thereof; as component (b), cellulose acetate phthalate, hydroxypropyl methylcellulose phthalate, polyvinyl acetate phthalate, methacrylic acid ester copolymer, hydroxypropyl methylcellulose acetate succinate, shellac, cellulose acetate trimellitate and mixtures thereof; and as component (c), polyvinylpyrrolidone, hydroxypropyl cellulose, hydroxypropyl methylcellulose, polyethylene glycol having a molecular weight of from 1700 to 20,000, polyvinyl alcohol and monomers therefore and mixtures thereof. Optionally, the coating comprises: 35 to 75% by weight of component (a); 2-20% by weight of component (b); and 15-40% by weight of component (c). In yet a further variant, the coating comprises: polyethylene glycol having a molecular weight of from 1700 to 20, 000 15 to 40%; ethylcellulose 45 to 65%; methacrylic acid: acrylic; and, acid ethylester 1:1 copolymer 4 to 20%.

In combination with the coatings described above, and in another variant of the invention, the core comprises the following components: (a) at least 35% by weight of a matrix polymer which is insoluble at a pH of from 1 to 7.5 and is composed of ethyl cellulose, a quaternary ammonium acrylic or methacrylic polymer, an acrylic or a methacrylic ester copolymer or a mixture thereof which contributes to the control of the release of the active ingredient in the stomach and intestines. Component (b) is from 1 to 30% of an enteric polymer which is substantially insoluble at a pH of from 1 to 4, sufficient to delay the release of the active ingredient in the stomach. Component (b) is soluble at a pH of from 6 to 7.5 so as not to substantially delay release in the intestines; (c) from 1 to 60% of a compound soluble at a pH of from 1 to 4, and sufficient to enable initiation of release of the active ingredient in the stomach. The percentages are by weight based on the total weight of components (a), (b), and (c). The ratio of the components (a), (b), and (c) in the coating are such that a dose of the pellet composition delivers to the patient a therapeutically effective amount of the active ingredient over the course of the predetermined interval, so as to maintain an active ingredient blood level at steady state of at least 75% of maximum blood level for more than approximately 4 hours and so that the time at which the active ingredient reaches its maximum concentration is between about 4 and about 30 hours. Optionally, the time at which the active ingredient reaches its maximum concentration is between about 4 and about 12 hours.

In yet another variant, the core comprises a therapeutically effective amount of a resinate of morphine. In addition to said resinate, the coating on the core comprises the following components: (a) at least 35% by weight of a matrix polymer which is insoluble at a pH of from 1 to 7.5 and is composed of ethyl cellulose, a quaternary ammonium acrylic or methacrylic polymer, an acrylic or a methacrylic ester copolymer or a mixture thereof. These components contribute to the control of the release of the active ingredient in the stomach and intestines. The coating comprises component (b) which is from 1 to 30% of an enteric polymer that is substantially insoluble at a pH of from 1 to 4, and sufficient to delay the release of the active ingredient in the stomach. This component is soluble at a pH of from 6 to 7.5 so as not to substantially delay release in the intestines. Component (c) is from 1 to 60% of a compound soluble at a pH of from 1 to 4, and is sufficient to enable initiation of release of the active ingredient in the stomach. The percentages by weight are based on the total weight of components (a), (b), and (c); and, the ratio of the components (a), (b), and (c) in the coating are such that a dose of the pellet composition delivers to the patient a therapeutically effective amount of the active ingredient over the course of the predetermined interval, so as to maintain an active ingredient blood level at steady state of at least 75% of maximum blood level for more than approximately 4 hours and so that the time at which the active ingredient reaches its maximum concentration is between about 4 and about 30 hours.

In yet a further variant, the oral pharmaceutical dosage form optionally comprises an aversive agent. The aversive agent is selected from the group consisting of capsaicin, capsico, capsacutin, dihydrocapsaicin, nordihydrocapsaicin, homocapsaicin, homodihydrocapsaicin, capsaicinoids, gingerol, chemical mace, piperine, isochavicine, isopiperine, piperidine, chavicine, piperettine, zingerone, shogaol, valleral, an isovalleral, vanyllylamide, nonoyl vanyllamide, a vanyllylamide derivative, a synthetic derivative of a capsaicinoids, and a powder of: a Capsicum frutescens variety, a Capsicum anuum variety, a Capsicum chinense variety, a Capsicum baccatum variety, a Capsicum pubescens variety, a Capsicum species, a Piper migrum variety, a Piper longum variety, a Piper retrofractum variety, a Piper officinarum variety, a Piperaceae species, a Brassica juncea variety, a Brassica. nigra variety, a Sinapis alba variety, a Sinapis arvensis variety, a Zingiber officinale variety, and a Lactarius vellereus variety, and a mixture thereof. It is appreciated that other aversive agents can also be used.

The core comprises a drug which is a controlled substance in one variant of the invention. Optionally, the aversive agent and the controlled substance are loaded onto an ion exchange resin. It is also appreciated that the aversive agent is loaded onto the ion exchange resin, and the controlled substance is not loaded onto the ion exchange resin in another variant. In yet a further variant, the controlled substance is loaded onto the ion exchange resin, and the aversive agent is not loaded onto the ion exchange resin. In yet another manner to proceed with the invention, the controlled substance is loaded onto a first ion exchange resin, and the aversive agent is loaded onto an ion exchange resin different from the first ion exchange resin. The oral pharmaceutical dosage form is not susceptible to abuse by respiratory mucosal membrane administration, and it includes one or more aversive agents, and one or more resonates in yet another variant.

A respiratory irritant such as powdered chili peppers, or concentrated extracts of such products that contain capsaicin or capsaicin-like components, is incorporated into the solid oral dosage form of the controlled substance. When the oral dosage form is used as prescribed, i.e. swallowed whole, the irritant causes no aversive response. However, if the oral dosage form is rendered into a powder and inhaled, the irritant creates intense discomfort in the user, including coughing, dyspnea, rhinitis, nasal congestion, eye irritation, lachrymation, and sneezing. This intense discomfort has the effect of deterring a first group of people from using the inhalation route as a means of administration, i.e. it elicits an aversive response.

A bitter tasting agent such as denatonium benzoate (Bitrex®) or a sour tasting agent such as citric acid, is incorporated into the solid oral dosage form of the controlled substance. When the oral dosage form is used as prescribed, i.e. swallowed whole, the bitter or sour substance is provided in a form not to cause an aversive response. However, if the oral dosage form is chewed, the bitter or sour substance creates an intensely unpleasant taste. This unpleasant taste has the effect of deterring people from chewing the dosage form, i.e. it elicits an aversive response from the first group of people.

While the use of a resinate of the active ingredient reduces the amount of the active ingredient being extracted illicitly, some abusers may still choose to prepare and administer such an extract. The inclusion of the aversive agent in the present invention will result in the aversive agent being present in the extract, further reducing the motivation to abuse the formulation by the preparation of an illicit extract. If the presence of the aversive agent makes the formulation unpalatable, even when administered for therapeutic use then the therapeutic use of the dosage form will be compromised. In the current invention, this problem can be eliminated by using a resinate of the aversive agent, such that when the dosage form is abused by illicit extraction or chewing the aversive agent is release from the resinate, causing an aversive response. The combination of a resinate of the active ingredient together with either an aversive agent or a resinate of an aversive agent is therefore synergistic.

The present invention results in an unexpectedly large reduction in the potential for the abuse of drug formulations. It is effective against all known methods of abuse and is not limited to the subjective perceptions of the abuser. Prevention of abuse by injection of an illicit extract is counteracted by a poor extraction efficiency in the preparation of the illicit extracts. This is not subjective. Prevention of abuse by crushing and inhaling is counteracted by two mechanisms, namely slow release of the opiate from the resinate, and by the presence of an aversive agent. The former is not subjective. Prevention of abuse by chewing is counteracted by two mechanisms, namely slow release of the opiate from the resinate, and by the presence of an aversive agent. The former is not subjective. Prevention of abuse by the ingestion of an illicit extract is counteracted by two mechanisms, namely a poor extraction efficiency in the preparation of the illicit extract, and the presence of the aversive agent in the illicit extract. The former is not subjective. Additionally the presence of the aversive agent does not render the dosage form unpalatable when administered for therapeutic purposes.

Active ingredients useful in the practice of the invention codeine, dextromoramide, hydrocodone, hydromorphine, pethidine, methadone, morphine, oxycodone, dihydrocodeine, fenatanyl, and propoxyphene.

The opiate-ion exchange resin complex can by formulated into any of the oral dosage forms known in the art including, but not limited to, powders, tablets, transdermal patches, pills, and capsules. U.S. Pat. No. 5,378,474 and U.S. Pat. No. 6,066,339 (incorporated by reference as if fully set forth) give further examples of forms that can be used in the present invention.

The controlled substance-ion exchange resin complex can be prepared by any of the methods known in the art. The typical method, known to those skilled in the art, for loading ionizable substances onto an ion exchange resin to form the ionizable substance-ion exchange resin complex is to dissolve an acidic or basic, ionizable substance in water, and then mix it with a suitable ion exchange resin.

Aversive agents useful in the practice of this invention include, but are not limited to, respiratory irritants, bitter substances, and sour substances. Aversive agents useful in the practice of this invention are solids in one variant. The solid can be the agent in pure form or a solid containing the agent. Aversive agents useful in the practice of this invention are of natural or synthetic origin. One aspect of this invention is the use of capsaicinoids as an aversive agent which acts as a respiratory irritant to create an aversive response. Capsaicinoids are alkaloid substances which occur naturally in the fruit of various chile pepper plants. The principal capsaicinoids found in most pepper plants are capsaicin, dihydrocapsaicin, capsico, and capsacutin. The principal capsaicinoid is capsaicin. There can be multiple capsaicinoids in one pepper and different peppers have different concentrations of capsaicinoids. The production of capsaicinoids is a form of chemical defense against being eaten and thus acts naturally as an animal repellant. See, Smith, R. L., Ecology and Field Biology, p. 562 (3d Ed. 1980). Capsaicinoids are the chemicals responsible for the “hot” sensation associated with peppers. The hotness of the various capsicums is directly attributable to their capsaicinoid content. Capsaicinoids generate a spicy flavor in the mouth but are irritants when applied to mucous membranes.

Capsicum is the formal term used to refer to the dried ripe fruit of the various species of chili peppers.

Therapeutically, capsaicin is listed as a counterirritant (Merck Index, 9th Ed., p. 224). Capsicum has Generally Regarded as Safe (GRAS) status in the USA. Capsaicin, capsicum, and capsicum oleoresin have monographs in the US Pharmacopeia 24.

Respiratory irritants useful in the practice of this invention include, but are not limited to, pure compounds and mixtures of capsaicin, capsico, capsacutin, dihydrocapsaicin, nordihydrocapsaicin, homocapsaicin, homodihydrocapsaicin, capsaicinoids, gingerol, chemical mace, piperine, isochavicine, isopiperine, piperidine, chavicine, piperettine, zingerone, shogaol, valleral, isovallerals, vanyllylamide, nonoyl vanyllamide, vanyllylamide derivatives, synthetic derivatives of capsaicinoids, and extracts, capsicums, and powders of, Capsicum frutescens varieties, Capsicum anuum varieties, Capsicum chinense varieties, Capsicum baccatum varieties, Capsicum pubescens varieties, Capsicum species, Piper migrum varieties, Piper longum varieties, Piper retrofractum varieties, Piper officinarum varieties, Piperaceae species, Brassica juncea varieties, Brassica. nigra varieties, Sinapis alba varieties, Sinapis arvensis varieties, Zingiber officinale varieties, and Lactarius vellereus varieties and mixtures thereof.

Preferred respiratory irritants useful in the practice of the invention are pure compounds and mixtures of capsaicin, capsico, capsacutin dihydrocapsaicin, nordihydrocapsaicin, homocapsaicin, homodihydrocapsaicin, capsaicinoids, gingerol, piperine, isopiperine, piperidine, piperettine, zingerone, shogaol, valleral, isovallerals, vanyllylamide, vanyllylamide derivatives, and extracts, capsicums, and powders of, Capsicum frutescens varieties, Capsicum anuum varieties, Capsicum chinense varieties, Piper migrum varieties, Piper longum varieties, Piper retrofractum varieties, Piper officinarum varieties, Brassica juncea varieties, Brassica. nigra varieties, Sinapis alba varieties, Sinapis arvensis varieties, and Zingiber officinale varieties and mixtures thereof.

More preferred respiratory irritants useful in the practice of the invention are pure compounds and mixtures of capsaicin, capsacutin dihydrocapsaicin, nordihydrocapsaicin, homocapsaicin, homodihydrocapsaicin, capsaicinoids, gingerol, piperine, isopiperine, zingerone, shogaol, and vanyllylamide derivatives and mixtures thereof.

The use of capsaicin with cocaine is contra-indicated.

The amount of respiratory irritant useful in the practice of this invention is that which is sufficient to elicit an aversive response in the user when the irritant is inhaled through the respiratory mucosa but that which is not sufficient to elicit an aversive response or an adverse medical response in the user when the irritant is swallowed as a solid oral dosage form in the manner prescribed.

The nociceptive efficacy of the respiratory irritants varies greatly depending both on chemical structure of the active ingredient of the irritant, and the amount of active ingredient in the irritant. The following amounts of respiratory irritants are provided as examples. Effective amounts of other respiratory irritants can be determined using techniques well known to those skilled in the art.

The amount of the respiratory irritants capsaicin and dihydrocapsaicin is a combined total of 0.002-100 mg per dose in one variant of the invention.

The amount of the respiratory irritant zingerone is optionally 0.04-200 mg per dose. The amount of the respiratory irritant shogaol is optionally 0.04-200 mg per dose. The amount of the respiratory irritant piperine is optionally 0.04-200 mg per dose. The amount of the respiratory irritants capsicums of Capsicum annum, Capsicum frutescens, and Capsicum chinense is optionally 0.1-450 mg per dose. The amount of the respiratory irritants capsaicin and dihydrocapsiacin is optionally a combined total of 0.004-25 mg per dose. The amount of the respiratory irritant zingerone is optionally 0.2-150 mg per dose. The amount of the respiratory irritant shogaol is optionally 0.2- 150 mg per dose. The amount of the respiratory irritant piperine is optionally 0.2- 150 mg per dose. The amount of the respiratory irritants capsicums of Capsicum annum, Capsicum frutescens, and Capsicum chinense is optionally 0.4-350 mg per dose. The amount of the respiratory irritants capsaicin and dihydrocapsiacin is optionally a combined total of 0.02-15 mg per dose. The amount of the respiratory irritant zingerone is optionally 0.2-100 mg per dose. The amount of the respiratory irritant shogaol is optionally 0.2- 100 mg per dose. The amount of the respiratory irritant piperine is optionally 0.2-100 mg per dose. The amount of the respiratory irritants capsicums of Capsicum annum, Capsicum frutescens, and Capsicum chinense is optionally 0.6-250 mg per dose.

Bitter agents also create an aversive response. The use of bitter agents is particularly useful in preventing abuse of controlled substances by chewing the oral dosage form. Bitter agents useful in the practice of this invention include, but are not limited to, agaricic acid, benzyl acetate, brucine, brucine sulfate, caffeine, capsaicin, catechin, dadzein, denatonium benzoate (Bitrex®) and other denatonium salts, denatonium capsaicinate, denatonium chloride, denatonium saccharide, diethyl phthalate, epicatechin, genistein, gentian violet, gerianol, hydroxytyrosol, kashin, limonin, linalool, linalool acetate, methyl anthranilate, naringin, nobiletin, oleuropin, phenylethyl alcohol, polyphenols, quassin, quebracho, quercitin, quinine, quinine sulfate, quinine hydrochloride, sinensetin, sucrose benzoate, sucrose octaacetate, and tangeretin and mixtures thereof.

Bitter agents useful in the practice of this invention are, denatonium benzoate (Bitrex®) and other denatonium salts, denatonium capsaicinate, denatonium chloride, denatonium saccharide, limonin, linalool, linalool acetate, naringin, quassin, quercitin, sucrose benzoate, and sucrose octaacetate and mixtures thereof. Other bitter agents useful in the practice of this invention are denatonium benzoate (Bitrex®), denatonium capsaicinate, denatonium saccharide, and sucrose octaacetate and mixtures thereof. The amount of bitter agent useful in the practice of this invention is that which creates an aversive response in the abuser and/or a group of abusers.

Further, sour agents also create an aversive response. The use of sour agents is particularly useful in preventing abuse of controlled substances by chewing the oral dosage form. Sour agents useful in the practice of this invention include, but are not limited to, acidic organic compounds that contain one or more acidic protons per molecule and mixtures thereof. Sour agents useful in the practice of the invention are acidic organic compounds that contain two or more acidic protons per molecule and mixtures thereof. Other sour agents useful in the practice of the invention are citric acid and tartaric acid and mixtures thereof. The amount of sour agent useful in the practice of this invention is that which creates an aversive response in the abuser.

The controlled substance and aversive agent are incorporated into the dosage form using any of the methods known in the art for preparation of solid oral dosage forms. See, Remington's Pharmaceutical Sciences, 16^{th} Edition.

In one embodiment of the invention, the opiate-resin complex, with or without aversive agent is incorporated into a multiparticulate dosage form. A multiparticulate dosage form comprises an inert core upon which is deposited a coating. The coating comprises one or more layers. The layers may have different compositions. Any or all of the layers may contain the opiate-resin complex. Such coatings are selected to obtain the desired release rate profile of the active ingredient, and can include layers that are permeable, non-permeable, water soluble, partially water soluble, and coatings having water solubility that is pH dependent.

The core may be of any suitable type. A sugar core may be used. The size and amount of the core may vary substantially from approximately 0.1 mm to 1.7 mm depending upon the amount of active ingredient to be included. Accordingly, the core may vary from approximately 5 to 99% by weight, preferably 40 to 90% by weight, depending on the potency of the active ingredient. The core may be of such a diameter to provide a final coated core having a diameter of approximately 0.2 mm to 2 mm.

Further, different combinations of aversive agents can be combined in the same dosage form. For example, if it is desired to reduce the potential for abuse via both inhalation and chewing, it may be desirable to combine both a respiratory irritant and a bitter agent in the same formulation. Further, combination of avervise agents can sometime have a synergistic effect, such that the combination has a greater effect than the sum of the individuals taken separately. Still further, people have different responses to taste, such that a mixture of aversive agents may be needed to be effective in a larger fraction of the population.

In addition to the controlled substance and aversive agent, excipients are used in the manufacture of the oral dosage forms of the present invention. Excipients useful in the practice if this invention include but are not limited to preservatives, viscosity agents, fillers, lubricants, glidants, disintegrants, binders, and encapsulants.

Preferred preservatives include, but are not limited to, phenol, alkyl esters of parahydroxybenzoic acid, o-phenylphenol benzoic acid and the salts thereof, boric acid and the salts thereof, sorbic acid and the salts thereof, chlorobutanol, benzyl alcohol, thimerosal, phenylmercuric acetate and nitrate, nitromersol, benzalkonium chloride, cetylpyridinium chloride, methyl paraben, and propyl paraben. Particularly preferred are the salts of benzoic acid, cetylpyridinium chloride, methyl paraben and propyl paraben. The compositions of the present invention generally include from 0-2% preservatives.

Preferred viscosity agents include, but are not limited to, methylcellulose, sodium carboxymethylcellulose, hydroxypropyl-methylcellulose, hydroxypropylcellulose, sodium alginate, carbomer, povidone, acacia, guar gum, xanthan gum and tragacanth. Particularly preferred are methylcellulose, carbomer, xanthan gum, guar gum, povidone, sodium carboxymethylcellulose, and magnesium aluminum silicate. Compositions of the present invention include 0-25% viscosity agents.

Preferred fillers include, but are not limited to, lactose, mannitol, sorbitol, tribasic calcium phosphate, dibasic calcium phosphate, compressible sugar, starch, calcium sulfate, dextro and microcrystalline cellulose. The compositions of the present invention contain from 0-75% fillers.

Preferred lubricants include, but are not limited to, magnesium stearate, stearic acid, and talc. The pharmaceutical compositions of the present invention include 0-2% lubricants.

Preferred glidants include, but are not limited to, talc and colloidal silica. The compositions of the present invention include from 0-5% glidants.

Preferred disintegrants include, but are not limited to, starch, sodium starch glycolate, crospovidone, croscarmelose sodium, polacrilin potassium, and microcrystalline cellulose. The pharmaceutical compositions of the present invention include from 0-30% disintegrants.

Preferred binders include, but are not limited to, acacia, tragacanth, pregelatinized starch, gelatin, povidone, ethylcellulose, polyvinyl pyrrolidone, hydroxypropyl cellulose, hydroxypropyl methylcellulose, methylcellulose and hydroxyethyl cellulose, sugars and mixtures thereof. The binding agent may be provided in the form of a granulating solution. An aqueous or organic solvent may be included. Methanol, ethanol of mixtures thereof may be used as solvents. The compositions of the present invention include 0.1-15% binders.

Encapsulants useful in the practice of the present invention include, but are not limited to permeable coatings, impermeable coatings, and controlled release coatings. Permeable coatings useful in this invention are well know to one skilled in the art and include Eudragit® RL, and Eudragit® RS (Rohm-Pharma Darmstadt, Germany). Non-permeable coatings useful in this invention are well known to one skilled in the art and include Aquacoat CPD (FMC Corporation, Philadelphia, Pa., USA), Eudragite E100, Eudragit® L100, Eudragit® S100 (Rohm-Pharma Darmstadt, Germany), Kollicoat® MA 30 DP (BASF Aktiengesellschaft, Ludwigshafen, Germany), Opadry light pink.

Controlled release coatings may be of any suitable type and include, cellulose acetate phthalate, hydroxypropyl methylcellulose phthalate (HPMCP), polyvinyl acetate phthalate, methacrylic acid copolymer, hydroxypropyl methylcellulose acetate succinate, Eudragit L100-55, Eudragit RL, Eudragit RS, ammonium methacrylate copolymer types A and B (as defined in US Pharmacopea/National Formulary),shellac, cellulose acetate trimellitate polyvinyl pyrrolidone, hydroxypropyl cellulose, hydroxypropyl methylcellulose, polyethylene glycol, polyvinyl alcohol ethylcellulose, acrylic and/or methacrylic ester polymers, polymers or copolymers of acrylates or methacrylates having a low quaternary ammonium content, and mixtures thereof.

Plastizers for use with coatings useful in the practice of the invention include but are not limited to, diethyl phthalate, triethyl citrate, triethyl acetyl citrate, tributyl citrate, 1,2-propylene glycol, polyethylene glycols, and triacetin. Dosage forms of this invention may also include pharmaceutically acceptable bio-enhancers that enhance the passage of the active ingredient through the rate-controlling polymer coat or through the tissue in the gastrointestinal tract (GIT). Preferably, the bio-enhancer is an organic acid, a pharmaceutically acceptable salt, a GIT absorption enhancer or a combination thereof. Suitable bio-enhancers include but are not limited to adipic acid, ascorbic acid, citric acid, malic acid, succinic acid, tartaric acid, lactic acid, fumaric acid, monopotassium citrate, potassium acid tartrate, sodium fumarate, sodium dihydrogen phosphate, sodium bisulfate, sodium metabisulfate or combinations thereof.

Dosage forms of the present invention are immediate release or modified release. Specifically, the dosage form of the present invention renders the controlled substance unable to deliver the desired non therapeutic effect, i.e., the drug-induced high.

Ion exchange resins useful in the practice of the present invention are cation exchange resins. Preferably, the resins are suitable for human ingestion. Cation exchange resins include, but are not limited to, styrenic strongly acidic cation exchange resins with sulfonic or phosphonic acid functionalities having a weight capacity of 0.1 to 8 meq/g; and styrenic weakly acidic cation exchange resins with carboxylic or phenolic acid functionalities having a weight capacity of 0.1 to 8.5 meq/g; and acrylic or methacrylic weakly acidic cation exchange resins with a carboxylic or phenolic acid functionality with a weight capacity of 0.1 to 14 meq/g, that are suitable for human and animal ingestion.

Other cation exchange resins include, but are not limited to, styrenic weakly acidic cation exchange resin with a phenolic functionality with a weight capacity of 0.1 to 8.5 meq/g; and a styrenic strongly acidic cation exchange resin with a sulfonic acid functionality with weight capacity of 0.1 to 8 meq/g, or a methacrylic weakly acidic cation exchange resin with a carboxylic acid functionality with weight capacity of 0.1 to 12 meq/g.

Ion exchange resins useful in the practice of this invention have a moisture content between 0% and the water retention capacity of the resin.

Ion exchange resins useful in this invention have particle size from 0.0005 mm to 1 mm. Cation exchange resins useful in the practice of this invention are in the acid form, or salt form, or partial salt form.

The following non limiting examples illustrate the present invention:

### EXAMPLE 1

Preparation of an oxycodone/Bitrex-ion exchange resin complex: 40 g of oxycodone hydrochloride, a Schedule II controlled substance, and 4 g of denatonium benzoate (available as Bitrex® from Macfarlan Smith, Edinburgh, UK) are dissolved in 2000 ml of water. 110 g of a powdered cation exchange resin with sulfonic acid functionality in the sodium form is then added to this solution, and the resulting mixture is shaken for at least 12 hours. The mixture is filtered using a Buchner funnel with a filter capable of retaining particles >0.003 mm. The wet-cake is washed in place with 1000 ml of water. The wet-cake is dried in a vacuum oven at 60° C. for 15 hours, or until constant weight is reached, to give the oxycodone/Bitrex®-ion exchange resin complex.

### EXAMPLE 2

Preparation of a hydromorphone-ion exchange resin complex: 40 g of hydromorphone hydrochloride is dissolved in 2000 ml of water. 110 g of a powdered cation exchange resin with sulfonic acid functionality in the sodium form is then added to this solution, and the resulting mixture is shaken for at least 12 hours. The mixture is filtered using a Buchner funnel with a filter capable of retaining particles >0.003 mm. The wet-cake is washed in place with 1000 ml of water. The wet-cake is dried in a vacuum oven at 60° C. for 15 hours, or until constant weight is reached, to give the hydromorphone-ion exchange resin complex.

### EXAMPLE 3

Preparation of a morphine-ion exchange resin complex: 103 g of morphine free base is dissolved in 2000 ml of aqueous ethanol. 100 g of a powdered cation exchange resin with sulfonic acid functionality in the hydrogen form is then added to this solution, and the resulting mixture is shaken for at least 12 hours. The mixture is filtered using a Buchner funnel with a filter capable of retaining particles >0.003 mm. The wet-cake is washed in place with 1000 ml of water. The wet-cake is dried in a vacuum oven at 60° C. for 15 hours, or until constant weight is reached, to give the morphine-ion exchange resin complex. Each gram of the complex contains the equivalent of approximately 580 mg of morphine hydrochloride

### EXAMPLE 4

Preparation of a Bitrex®-ion exchange resin complex: the procedure of Example 2 is repeated except that the hydromorphone hydrochloride is replaced with 20 g of denatonium benzoate (available as Bitrex® from Macfarlan Smith, Edinburgh, UK).

### EXAMPLE 5

| Composition | |
|---|---|
| Oxycodone/Bitrex-ion exchange resin complex (Example 1) | 320.0 g |
| Lactose (ground) | 35.0 g |
| Colloidal silica | 3.0 g |
| Polyvinylpyrrolidone | 3.0 g |
| Microcrystalline cellulose | 40.0 g |
| Corn starch | 69.0 g |

All the solid ingredients are passed through a 0.6 mm sieve and mix together. The mixture is used to make 9 mm diameter tablets by compression. Each tablet weighs 230 mg and contains an amount of active ingredient equivalent to 40 mg of oxycodone hydrochloride.

### EXAMPLE 6

1500 g of the hydromorphone-ion exchange resin complex obtained in Example 2 is blended with 130 g of Bitrex®-ion exchange resin complex obtained in Example 3. The mixture is filled into 10,000 size 1 capsules. Each capsule contains an amount of active ingredient equivalent to 40 mg of hydromorphone hydrochloride.

### EXAMPLE 7

| | |
|---|---|
| First coating | |
| Morphine resinate of Example 3 | 355 g |
| Sugar cores | 722 g |
| Hydroxypropylmethylcellulose | 14 g |
| Ethanol | 881 g |
| Water | 105 g |
| Second coating | |
| Polyethylene glycol | 47 g |
| Ethylcellulose | 90 g |
| Diethylphthalate | 19 g |
| | 20 g |
| Talc | 88 g |
| Ethanol | 723.9 g |

A solution of 14 g of hydroxypropylmethylcellulose in 881 g of ethanol and 105 g of water is prepared. To this is added 355 g of the morphine resinate of Example 3. 722 g of sugar cores are placed in a rotor fluid bed machine and coated with the resinate suspension. The wet product so formed is then dried in a suitable drier for one hour to give the intermediate product. 47 g of polyethylene glycol, 90 g of ethylcellulose, 19 g of diethylphthalate, 20 g of methacrylic acid:acrylic acid ethylester 1:1 copolymer, and 88 g of talc are dispersed in 724 g of ethanol. This mixture is then sprayed onto the intermediate product in a rotary fluid bed coating apparatus to produce the final coated particles.

### EXAMPLE 8

A Caucasian male addict, age 48, uses a teaspoonful of a solution of common salt in water to prepare an illicit extract from a 40 mg oxycodone tablet, as prepared in Example 5. After filtering the extract he then injects it intravenously. Because the extraction is inefficient, the non-therapeutic effect, i.e., the drug-induced high, is not obtained.

### EXAMPLE 9

A Caucasian female addict, age 27, weighing 45 kg crushes two 40 mg oxycodone tablets, as prepared in Example 5, into a powder and administers the powder through her nasal cavity. The non-therapeutic effect, i.e. the drug-induced high, is not obtained.

### EXAMPLE 10

At a party a Caucasian male recreational drug user, aged 17, weighing 65 kg is offered two 40 mg oxycodone tablets, as prepared in Example 5, and chews them. The non-therapeutic effect, i.e. the drug-induced high, is not obtained, and the taste of the tablet is highly objectionable.

### EXAMPLE 11

An otherwise healthy Caucasian male, age 48, suffering from post-operational pain related to a severe laceration of a finger follows the instructions of his physician and swallows one oxycodone tablet, as prepared in Example 5. He perceives no bitter taste and experiences the full therapeutic response.

### EXAMPLE 12

An Asian female recreational drug user, aged 28, weighing 55 kg, breaks open and empties a hydromorphone capsule, as prepared in Example 6, and uses a shot-glass of a solution of common salt to prepare an illicit extract. She attempts to drink the extract, but the taste is highly objectionable and she does not finish drinking the extract. The non-therapeutic effect, i.e. the drug-induced high, is not obtained.

### EXAMPLE 13

24.46 g of oxycodone free base was dissolved in 660 g of 50% aq ethanol. 38.9 g of a powdered cation exchange resin with sulfonic acid functionality in the hydrogen form was then added and the mixture was stirred at room temperature for 15 hours. The slurry was filtered and the filter cake was washed with 4×350 g of 95% ethanol, then allowed to air dry, and finally dried in a vacuum oven at 60° C. Samples of the filtrate were analyzed for oxycodone. No oxycodone was detected. The oxycodone-resin complex contained 0.403 g of oxycodone per gram of complex.

1 g of the complex was mixed with 5 g of water and the mixture was shaken overnight. The mixture was centrifuged for 15 minutes. A sample of the clear supernatent was then analyzed for oxycodone. No oxycodone was detected. The limit of detection of the analytical method was 1 mg/l. This result demonstrates that the solubility of the oxycodone-resin complex in water is <1 in 1,000,000.

### EXAMPLE 14

One tablet of Percocet™, (5 mg oxycodone hydrochloride and 325 mg acetaminophen) is crushed and mixed with 10 ml of water. After approximately 20 minutes the mixture is cooled to 2° C. and allowed to sit for one hour. This has the effect of precipitating most of the dissolved acetaminophen. The mixture is then filtered and the filtrate analyzed for oxycodone. The extract is found to contain the equivalent of approximately 4.5 mg of oxycodone hydrochloride.

The procedure is then repeated using one tablet of Percocet™ in which the oxycodone hydrochloride is replaced with an equivalent amount of the oxycodone-resin complex of Example 13. The extract is found to contain no oxycodone.

We claim:

1. An oral pharmaceutical dosage form comprising, in combination, a core, and a coating surrounding the core comprising a resinate of an opiate,
whereby the pharmaceutical oral dosage form is not subject to abuse.

2. The oral pharmaceutical dosage form of claim 1 in which coating is an extended drug release coating.

3. An oral pharmaceutical dosage form of claim 1 in which the coating on the core further comprises the following components:
(a) from 1 to 85% by weight of a matrix polymer which is insoluble at a pH of from 1 to 7.5 and contributes to the control of the rate of release of the active ingredient in the stomach and intestines;
(b) from 1 to 30% of an enteric polymer which is substantially insoluble at a pH of from 1 to 4, sufficient to delay the release of the active ingredient in the stomach, but which is soluble at a pH of from 6 to 7.5 so as not to substantially delay release in the intestines;
(c) from 1 to 60% of a compound soluble at a pH of from 1 to 4, sufficient to enable initiation of release of the active ingredient in the stomach; the percentages being by weight based on the total weight of components (a), (b), and (c); the ratio of the components (a), (b), and (c) in the coating being such that a dose of the pellet composition delivers to the patient a therapeutically effective amount of the active ingredient over the course of the predetermined interval, so as to maintain an active ingredient blood level at steady state of at least 75% of maximum blood level for more than approximately 4 hours and so that the time at which the active ingredient reaches its maximum concentration is between about 4 and about 30 hours.

4. The oral pharmaceutical dosage form of claim 1 in which the opiate is selected from the group consisting of codeine, dextromoramide, hydrocodone, hydromorphine, pethidine, methadone, morphine, oxycodone, difydrocodeine, fentanyl, and propoxyphene.

5. The oral pharmaceutical dosage form of claim 1 wherein the coating further comprises: as component (a), ethyl cellulose, a quaternary ammonium acrylic or methacrylic polymer, an acrylic or a methacrylic ester copolymer or a mixture thereof; as component (b), cellulose acetate phthalate, hydroxypropyl methylcellulose phthalate, polyvinyl acetate phthalate, methacrylic acid:acrylic acid ester copolymer, hydroxypropyl methylcellulose acetate succinate, shellac, cellulose acetate trimellitate and mixtures thereof; and as component (c), polyvinylpyrrolidone, hydroxypropyl cellulose, hydroxypropyl methylcellulose, polyethylene glycol having a molecular weight of from 1700 to 20,000, polyvinyl alcohol and monomers therefor and mixtures thereof.

6. The oral pharmaceutical dosage form of claim 6 wherein the coating comprises: 35 to 75% by weight of component (a); 2-20% by weight of component (b); and 15-40% by weight of component (c).

7. The oral pharmaceutical dosage form of claim 7 wherein the coating comprises up to 50% of plasticizer selected from diethyl phthalate, triethyl citrate, triethyl acetyl citrate, triethyl acetin, tributyl citrate, polyethylene glycol having a molecular weight of from 200 to less than 1700 or glycerol and up to 75% of a filler selected from silicon dioxide, titanium dioxide, talc, alumina, starch, kaolin, polacrilin potassium, powdered cellulose and microcrystalline cellulose and mixtures thereof, the percentages being based on the total weight of the coating.

8. The oral pharmaceutical dosage form of claim 7 wherein the coating contains: component (a) 35 to 70%; component (b) 4 to 20%; component (c) 15 to 35%; and, plasticizer 4 to 30%.

9. The oral pharmaceutical dosage form of claim 8 wherein the coating comprises: as component (a), ethyl cellulose, a quaternary ammonium acrylic or methacrylic polymer, an acrylic or a methacrylic ester copolymer or a mixture thereof; as component (b), cellulose acetate phthalate, hydroxypropyl methylcellulose phthalate, polyvinyl acetate phthalate, methacrylic acid ester copolymer, hydroxypropyl methylcellulose acetate succinate, shellac, cellulose acetate trimellitate and mixtures thereof; and as component (c), polyvinylpyrrolidone, hydroxypropyl cellulose, hydroxypropyl methylcellulose, polyethylene glycol having a molecular weight of from 1700 to 20,000, polyvinyl alcohol and monomers therefore and mixtures thereof.

10. The oral pharmaceutical dosage form of claim 1 in which the oral pharmaceutical dosage form comprises an aversive agent.
